(19) United States
(12) Patent Application Publication
(10) Pub. No.: US 2002/0188316 A1
(43) Pub. Date: Dec. 12, 2002

(54) Gripping forceps

(75) Inventor: Andreas Dingler, Niefern-Oeschelbronn (DE)

Correspondence Address: AKIN, GUMP, STRAUSS, HAUER & FELD, L.L.P., ONE COMMERCE SQUARE, SUITE 2200, 2005 MARKET STREET, PHILADELPHIA, PA, 19103, US

(73) Assignee: Richard Wolf GmbH

(21) Appl. No.: 10/163,854

(22) Filed: Jun. 5, 2002

(30) Foreign Application Priority Data
Jun. 6, 2001 (DE) 101 27 506:4

(51) Int. Cl. A61B 17/28

(52) U.S. Cl. 606/205; 606/207

(57) ABSTRACT

The invention relates to a gripping forceps with two jaw parts forming a forceps jaw and comprising arcuate gripping surfaces, of which at least one is pivotable relative to the other for opening and closing the forceps jaw. For gripping the tissue in a manner such that no injuries occur, according to the invention it is envisaged that the one gripping surface of the one jaw part is curved convexly and the other gripping surface of the other jaw part is curved concavely, in a manner such that with a closed forceps jaw both gripping surfaces bear against one another.

### BACKGROUND OF THE INVENTION

The invention relates to a gripper forceps with two jaw parts which form a forceps jaw and comprise arcuate gripping surfaces, and of which at least one is pivotable relative to the other for opening and closing the forceps jaw.

Gripping forceps of this type have been known for a long time in the state of the art for surgical application. For example the U.S. Pat. No. 5,964,780 discloses such a forceps which preferably is used in minimal-evasive surgery. By closing the open forceps jaw, tissue may be gripped and held. The two parts of the forceps jaw are at the same time designed such that the two gripping surfaces according to one embodiment form are configured flat. There is also possible a design with which the two jaw parts are configured such that in the lateral view there results a wave-shaped course of the contact regions of the jaw mouth.

From the U.S. Pat. No. 5,498,292 there is known another configuration of a surgical forceps. Here there cooperate two jaw parts pivotable relative to one another, which may be designed beak-shaped. For the improved gripping of tissue the contact regions of the forceps jaw may be provided with saw-tooth-shaped profilings.

It has been shown to be disadvantageous with the previously known surgical gripper forceps that with large forceps forces, which are required for a secure gripping of tissue, the tissue is subjected to a correspondingly high loading. With this there exists the danger that the tissue is damaged. Furthermore the previously mentioned gripper forceps are not suited for securely holding surgical suture needles.

### BRIEF SUMMARY OF THE INVENTION

It is therefore the object of the invention to further develop a gripper forceps of the initially mentioned type in a manner such that these disadvantages may be avoided. It is therefore to be possible with the forceps to muster a maximal holding force with a minimal danger of damage of the tissue to be gripped. With this a secure holding of tissue is to be made possible without this being subjected to a large mechanical loading. Furthermore it is also to be possible with the forceps to hold an arc-shaped needle, in particular a suture needle. The forceps is therefore to be able to be used as a tissue gripping forceps as well as a needle holder.

The solution of this object by the invention is characterized in that the one gripping surface of the one jaw part is curved convexly and the other gripping surface of the other jaw part is curved concavely, in a manner such that with a closed forceps jaw both gripping surfaces bear against one another.

According to a further formation it is envisaged that both gripping surfaces are formed spherically. At the same time in particular the one gripping surface is formed by a ball section and the other gripping surface by a spherical annular surface which runs around a receded recess into which on closing the forceps jaw tissue may engage or penetrate, by which means the holding of the tissue becomes more secure.

Both gripping surfaces are preferably designed smooth, thus are not provided with a profiling.

If the forceps is designed such that one jaw part is unmovably arranged relative to the forceps base body, preferably this jaw part comprises the concavely shaped gripping surface. Furthermore it may also be envisaged for both jaw parts to be arranged pivotable relative to the forceps base body.

The gripper forceps according to the invention which for example may be used as an auxiliary instrument or forceps insert for endoscopy thus has the following advantages: a damage to the tissue which is held by the two jaw parts of the forceps is effectively prevented. The surface pressing onto the tissue is largely homogeneous by way of the suggested configuration so that the tissue is looked after. The differing curvature of the gripping surfaces has the effect that the tissue may be securely held. At the same time by way of the suggested shape there is not only a friction fit on closure of the jaw part but also a positive fit. It is also possible to securely hold small arcuate surgical sewing needles with the gripping forceps. For alternately holding tissue and needles it is not necessary for the forceps jaw parts to be exchanged.

### BRIEF DESCRIPTION OF THE DRAWINGS

In the drawing there is shown one embodiment example for a gripping forceps according to the invention. There are shown in

FIG. 1 the distal end section of a surgical gripping forceps with an opened forceps jaw in a lateral view and

FIG. 2 the forceps according to FIG. 1 without the one pivotable jaw part in a plan view.

### DETAILED DESCRIPTION OF THE INVENTION

The end section of the gripping jaw 1 is formed by the forceps jaw 2 which comprises a first pivotable jaw part 5 and a second stationary jaw part 6.

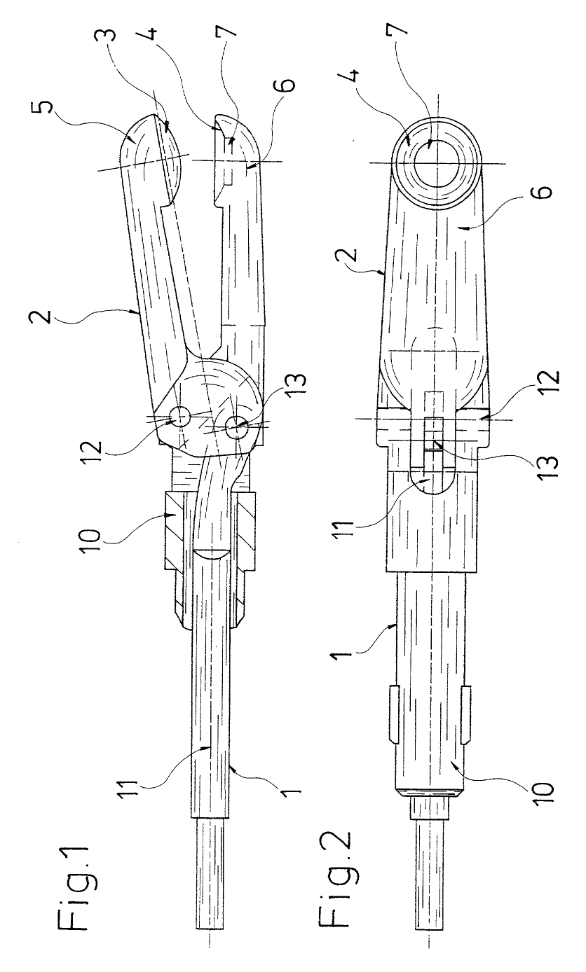

The actuation of the jaw part 2 is effected in a known manner. With this an axially displaceable actuation element 111 is displaced in the inside of a forceps shank and a hollow cylindrical formed forceps base body 10. The displacement movement is created by actuation of a handle which is not shown. As can be seen in FIG. 1 the jaw part 5 pivotably mounted by way of a stationary joint 12 with an axial displacement to the actuation element 11 is moved relative to the stationary jaw part 6. The actuation element 11 for this is formed bent at an angle at its distal end region and is fastened to the jaw part 5 by way of a joint 13.

Both jaw parts 5 and 6 comprise in each case an atraumatically formed gripping surface 3 and 4. The first gripping surface 3 at the same time in the embodiment example is designed convexly as the surface of a ball section. The second gripping surface 4 for this is formed complementarily concavely, i.e. in the closed condition of the forceps jaw 2 both gripping surfaces 3, 4 bear on one another.

The second gripping surface 4 comprises a * recess 7. The effectively active gripping surface between both jaw parts 5 and 6 or between the two gripping surfaces 3 and 4 is thus represented as an annular spherical surface. Thus tissue may be securely but atraumatically gripped, not least as both gripping surfaces 3, 4 are designed smoothly.

With the suggested design of the gripping surfaces 3 and 4 a secure holding of surgical suture needles is possible without any problem. With these arc-like needles it is mostly the case of elastic or flexible needles which on closing the forceps jaw by way of elastic deformation where required may also be adapted to the course of the gripping surfaces 3, 4 of the forceps.

The concept of the design of the two gripping surfaces 3, 4 of the jaw parts 5, 6 as specially arcuate surfaces fitting with one another is just as applicable when it is envisaged for both jaw parts 5, 6 to be designed pivotable relative to the forceps base body 10, as it is also possible according to an alternative formation.

1. A gripping forceps with two jaw parts forming a forceps jaw and comprising arcuate gripping surfaces, of which at least one is pivotable relative to the other for opening and closing the forceps jaw, wherein the one gripping surface of the one jaw part is curved convexly and the other gripping surface of the other jaw part is curved concavely, in a manner such that with a closed forceps jaw both gripping surfaces bear against one another.

2. A gripping forceps according to claim 1, wherein both gripping surfaces are formed spherically.

3. A gripping surface according to claim 2, wherein the one gripping surface is formed by a ball section and the other gripping surface by a spherical annular surface which runs around a receded recess.
